(19) United States
(12) Patent Application Publication
(10) Pub. No.: US 2011/0034380 A1
(43) Pub. Date: Feb. 10, 2011

(54) COMBINATION OF A SELECTIVE PPAR-GAMMA MODULATOR AND AN INCRETIN FOR THE TREATMENT OF DIABETES AND OBESITY

(76) Inventors: Dennis Lanfear, Portola Valley, CA (US); Linda Higgins, Palo Alto, CA (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/850,883

(22) Filed: Aug. 5, 2010

Related U.S. Application Data
(60) Provisional application No. 61/231,499, filed on Aug. 5, 2009.

(51) Int. Cl. A61K 31/47; A61K 38/26; A61P 3/10; A61P 3/04; A61P 3/06; A61P 9/10; A61P 9/12; A61P 3/00; A61P 15/08

(52) U.S. Cl. 514/7.2; 514/312; 514/11.7

(57) ABSTRACT

The present invention relates to pharmaceutical compositions and methods for the treatment of diabetes, obesity or disorders related to diabetes or obesity. The compositions comprise a combination of a selective PPARγ modulator and an incretin. The methods include the administration of the combination of a selective PPARγ modulator and an incretin.

### 1. FIELD OF THE INVENTION

The present invention relates to combinations of a selective PPARγ (PPAR-gamma) modulator and an incretin, pharmaceutical compositions and kits containing such combinations, and methods of using such combinations for the treatment of diabetes, pre-diabetes and obesity and/or disorders related to diabetes, pre-diabetes or obesity.

### 2. BACKGROUND OF THE INVENTION

Diabetes is now considered to be a worldwide epidemic. Globally, the number of people with diabetes is expected to rise from the current estimate of 150 million to 220 million in 2010 and 300 million in 2025. In the United States, it is estimated that as of 2002, 18.2 million people (6.3% of the total population) were diabetic.

Diabetes refers to a disease process derived from multiple causative factors and characterized by elevated levels of plasma glucose or hyperglycemia in the fasting state or after administration of glucose during an oral glucose tolerance test. Persistent or uncontrolled hyperglycemia is associated with increased and premature morbidity and mortality and thus presents significant public health concerns. Pre-diabetes is a condition in which a fasting plasma glucose test and/or an oral glucose tolerance test provide readings that are elevated, but not quite diabetic. Patients exhibiting pre-diabetes glucose readings are considered to be at higher risk for developing diabetes.

There are two generally recognized forms of diabetes. In type 1 diabetes, or insulin-dependent diabetes mellitus (IDDM), patients produce little or no insulin, the hormone which regulates glucose utilization. In type 2 diabetes (T2DM), or noninsulin dependent diabetes mellitus (NIDDM), patients often have plasma insulin levels that are the same or even elevated compared to nondiabetic subjects. However, these patients have developed resistance to the insulin stimulating effect on glucose and lipid metabolism in the main insulin-sensitive tissues.

Approved treatments for T2DM have recognized limitations. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g. tolbutamide and glipizide) or meglitinide, which stimulate the pancreatic 13 cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. The biguanides phenformin and metformin increase insulin sensitivity resulting in some correction of hyperglycemia, although both have known side effects. Metformin has fewer side effects than phenformin and is often prescribed for the treatment of T2DM.

Peroxisome proliferator-activated receptor γ (“PPARγ”) is one member of the nuclear receptor superfamily of ligand-activated transcription factors and has been shown to be expressed at particularly high levels in adipose tissue. Its expression is induced early during the course of differentiation of several preadipocyte cell lines. Additional research has now demonstrated that PPARγ plays a pivotal role in inducing adipocyte differentiation and maturation. PPARγ also regulates adiponectin which is involved in regulating energy homeostasis and adipocyte differentiation, which has been shown to be a critical step to be targeted for anti-obesity and diabetic conditions.

In view of the clinical importance of PPARγ, compounds that modulate PPARγ function, in particular those that can do so selectively, can be used for the development of new therapeutic agents for the treatment of diabetes. Potent selective modulators of PPARγ have been described, for example, in U.S. Pat. Nos. 6,200,995, 6,583,157, 6,653,332, and 7,041,691. One of these promising selective modulators, identified as compound 101, is in clinical development for diagnosis or therapeutic treatment of T2DM. A distinction is recognized in the literature between agents that act as selective modulators of PPARγ, and those that are understood to act as “full agonists of PPARγ.” Compound 101 is not a full agonist, but is instead a selective modulator of PPARγ that elicits a subset of the full spectrum of PPARγ response, whereas the thiazolidinedione (TZD) compounds rosiglatazone (Avandia®) and pioglitazone (Actos®) are considered full agonists of PPARγ. A selective modulator of PPARγ, such as compound 101, disclosed herein, can elicit the beneficial glucose and lipid lowering effects from activation of PPARγ, with little if any of the harmful side effects associated with the full agonists (e.g., weight gain, fluid retention, and loss of bone density.) The difference in mechanism and side effects between the full agonists of PPARγ and the selective modulators of PPARγ such as compound 101 are discussed in greater detail in “Higgins L S, Montzoros C S. “The development of INT131 as a Selective PPARγ Modulator: Approach to a Safer Insulin Sensitizer,” PPAR Research Volume 2008; Article ID 936906; and Zhang F, Lavan B E, and Gregoire F M, “Selective Modulators of PPARγ Activity: Molecular Aspects Related to Obesity and Side-Effects.” PPAR Research Volume 2007 Article ID 32696.

Incretins, and in particular, incretin mimetics (i.e., compounds that act like the natural hormones), are also under investigation as drugs that may be useful in the treatment of diabetes, and particularly, T2DM. The therapeutic utility of incretin mimetics for the treatment of T2DM is discussed, for example, in Ding X, Saxena N K, Lin S, Gupta N A, Anania F A. “Exendin-4, a glucagon-like protein-1 (GLP-1) receptor agonist, reverses hepatic steatosis in ob/ob mice”. Hepatology. 2006; 43(1):173-81: Tushuizen M E, Bunck M C, Pouwels P J, van Waesberghe J H, Diamant M, Heine R J. “Incretin mimetics as a novel therapeutic option for hepatic steatosis”. Liver Int. 2006; 26(8):1015-7, Fowler M J. “Diabetes Treatment, Part 3: Insulin and Incretins. Clinical Diabetes 2008; 26(1):35-39; Drucker D J. “Enhancing incretin action for the treatment of type 2 diabetes.” Diabetes Care 2003; 26:2929-2940; Turton M D, O'Shea D, Gunn I, Beak S A, Edwards C M, Meeran K, Choi S J, Taylor G M, Heath M M, Lambert P D, Wilding J P, Smith D M, Ghatei M A, Herbert J, Bloom S R “A role for glucagon-like peptide-1 in the central regulation of feeding.” Nature 1996; 379:69-72; Nauck M A, Bailer B, Meier J J. “Gastric inhibitory polypeptide and glucagon-like peptide-1 in the pathogenesis of type 2 diabetes.” Diabetes 2004 53 (Suppl. 3):S190-196.

The usefulness of incretins in the treatment of T2DM is based in part on the fact that incretins work to increase insulin secretion, among other action. There are two main incretin hormones in humans: glucagon like peptide-1 (GLP-1) and gastric inhibitory peptide (GIP). These hormones are secreted when food is consumed. Incretin-based drugs include exenatide (Byetta) approved in the U.S for the treatment of T2DM, and the GLP-1 mimetic Liraglutide (recently approved in Europe).

There is a continuing need for new methods of treating diabetes, obesity or disorders related to diabetes or obesity. The present invention addresses this problem by providing a combination therapy comprising a selective PPARγ modulator and an incretin for the treatment of diabetes, obesity or disorders related to diabetes or obesity.

### 3. SUMMARY OF THE INVENTION

The present invention provides combinations comprising a selective PPARγ modulator and an incretin, which are useful in the treatment of diabetes, obesity, or disorders related to diabetes or obesity. The combinations provide one or more clinical advantages over the use of a single agent alone, including but not limited to increased clinical efficacy and reduction in side effects such as weight gain, fluid retention and bone loss.

In certain embodiments, the selective PPARγ modulator of the combinations is compound 101 of formula I or a pharmaceutically acceptable salt, hydrate or polymorph thereof:

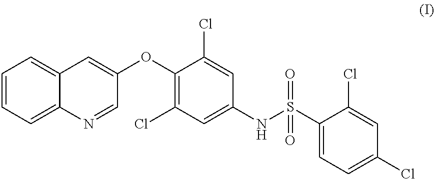

The incretins suitable for the purposes of the invention can be any naturally occurring or synthetic compounds that are gastrointestinal hormones, or their mimetics, that cause an increase in the amount of insulin released from the beta cells of the islets of Langerhans in response to food intake. Exemplary incretins are described in detail below.

In one embodiment, the incretin is selected from the group consisting of a glucagone-like peptide-1 (GLP-1) receptor agonist and glucose-dependent insulinotropic peptide (GIP) receptor agonist. In certain embodiments, the incretin is a GLP-1 receptor agonist selected from the group consisting of exenatide; a long-acting-release (LAR) variant of exenatide known as liraglutide; taspoglutide; CJC-1131; LY307161 SR; and AVE0010/ZP10.

The combinations of the present invention are useful in the treatment, of diabetes, obesity, or disorders related to diabetes or obesity. Disorders related to diabetes or obesity are described in detail below. Exemplary disorders related to diabetes or obesity include but are not limited to hyperglycemia, prediabetes, impaired glucose tolerance, impaired fasting glucose, dyslipidemia, hyperlipidemia, hypertriglyceridemia, hypercholesterolemia, low HDL levels, high LDL levels, atherosclerosis, hypertension, sleep apnea, polycystic ovarian syndrome, and metabolic syndrome.

The present invention also provides methods of treating these disorders comprising administering to a subject in need thereof a therapeutically effective amount of a compound of formula (I):

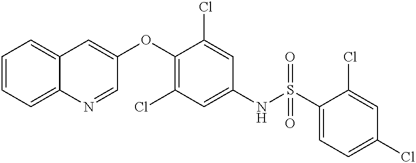

or a pharmaceutically acceptable salt, hydrate or polymorph thereof; and a therapeutically effective amount of an incretin.

The present invention provides a pharmaceutical composition comprising a compound of formula (I):

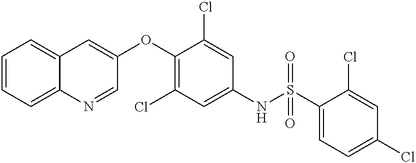

or a pharmaceutically acceptable salt, hydrate or polymorph thereof; and a therapeutically effective amount of an incretin.

The present invention also provides kits comprising compound 101 or a pharmaceutically acceptable salt, hydrate or polymorph thereof; and a therapeutically effective amount of an incretin.

### 4. DETAILED DESCRIPTION OF THE INVENTION

### 4.1 Definitions

The term “composition” as used herein is intended to encompass a product comprising the specified ingredients (and in the specified amounts, if indicated), as well as any product which results, directly or indirectly, from combination of the specified ingredients in the specified amounts. By “pharmaceutically acceptable” it is meant the diluent, excipient or carrier must be compatible with the other ingredients of the formulation and not deleterious to the recipient thereof.

The term “pharmaceutically acceptable salts” is meant to include salts of active compounds which are prepared with relatively nontoxic acids. Acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic; propionic; isobutyric; maleic; malonic; benzoic; succinic; suberic; fumaric; mandelic; phthalic; benzenesulfonic; toluenesulfonic, including p-toluenesulfonic, m-toluenesulfonic, and o-toluenesulfonic; citric; tartaric; methanesulfonic; and the like. Also included are salts of amino acids such as arginate and the like, and salts of organic acids like glucuronic or galactunoric acids and the like (see, for example, Berge et al. J. Pharm. Sci. 66:1-19 (1977)).

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The terms, “polymorphs” and “polymorphic forms” and related terms herein refer to crystal forms of a molecule. Different polymorphs may have different physical properties such as, for example, melting temperatures, heats of fusion, solubilities, dissolution rates and/or vibrational spectra as a result of the arrangement or conformation of the molecules in the crystal lattice. The differences in physical properties exhibited by polymorphs affect pharmaceutical parameters such as storage stability, compressibility and density (important in formulation and product manufacturing), and dissolution rates (an important factor in bioavailability). Polymorphs of a molecule can be obtained by a number of methods, as known in the art. Such methods include, but are not limited to, melt recrystallization, melt cooling, solvent recrystallization, desolvation, rapid evaporation, rapid cooling, slow cooling, vapor diffusion and sublimation.

Techniques for characterizing polymorphs include, but are not limited to, differential scanning calorimetry (DSC), X-ray powder diffractometry (XRPD), single crystal X-ray diffractometry, vibrational spectroscopy, e.g., IR and Raman spectroscopy, solid state NMR, hot stage optical microscopy, scanning electron microscopy (SEM), electron crystallography and quantitative analysis, particle size analysis (PSA), surface area analysis, solubility studies and dissolution studies.

The term, “solvate,” as used herein, refers to a crystal form of a substance which contains solvent. The term “hydrate” refers to a solvate wherein the solvent is water.

The term, “desolvated solvate,” as used herein, refers to a crystal form of a substance which can only be made by removing the solvent from a solvate.

The term “alkyl,” as used herein refers to monovalent saturated aliphatic hydrocarbyl groups particularly having up to about 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “lower alkyl” refers to alkyl groups having 1 to 6 carbon atoms. The term “alkyl” also includes “cycloalkyl” as defined below.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and 5, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH_{2-S}(O)—CH₃, —CH₂—CH_{2-S}(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Also included in the term “heteroalkyl” are those radicals described in more detail below as “heteroalkylene” and “heterocycloalkyl.”

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Particularly, an aryl group comprises from 6 to 14 carbon atoms.

The terms “treat”, “treating” or “treatment”, as used herein, refer to the reduction or amelioration of the progression, severity, and/or duration of a disorder or the eradication, reduction or amelioration of symptoms of a disorder, or the delay of the recurrence or onset of a disorder or one or more symptoms thereof in a subject that results from the administration of one or more compound.

The term “therapeutically effective amount” refers to the amount of the subject salt or polymorph that will elicit the biological or medical response of a tissue, system, animal or human that is being sought by the researcher, veterinarian, medical doctor or other clinician or that is sufficient to prevent development of or alleviate to some extent one or more of the symptoms of the disease being treated.

The term “incretin” is defined herein to include incretins, incretin analogs and incretin mimetics. It is not intended to include DPP-4 inhibitors.

The term “subject” is defined herein to include animals such as mammals, including, but not limited to, primates (e.g., humans), cows, sheep, goats, horses, dogs, cats, rabbits, rats, mice and the like. In preferred embodiments, the subject is a human.

As used herein, “diabetes” refers to type I Diabetes (juvenile diabetes), type II diabetes mellitus (non-insulin-dependent diabetes mellitus or T2DM), and pre-diabetes. Pre-diabetes is defined as a condition in which a fasting plasma glucose test and/or an oral glucose tolerance test provide readings that are elevated, but not considered diabetic.

The term “obesity” as used herein is a condition in which there is an excess of body fat. In certain embodiments, obesity is defined based on the Body Mass Index (BMI), which is calculated as body weight per height in meters squared kg/m². In some embodiments, an “obese subject” can be an otherwise healthy subject with a Body Mass Index (BMI) greater than or equal to 30 kg/m² or a subject with at least one co-morbidity with a BMI greater than or equal to 27 kg/m². In some embodiments, a “subject at risk of obesity” can be an otherwise healthy subject with a BMI of 25 kg/m² to less than 30 kg/m² or a subject with at least one co-morbidity with a BMI of 25 kg/m² to less than 27 kg/m².

The term “metabolic syndrome” as used herein is as defined by the Adult Treatment Panel III (ATP III; National Institutes of Health: Third Report of the National Cholesterol Education Program Expert Panel on Detection, Evaluation, and Treatment of High Blood Cholesterol in Adults (Adult Treatment Panel III), Executive Summary; Bethesda, Md., National Institutes of Health, National Heart, Lung and Blood Institute, 2001 (NIH pub. No 01-3670). Briefly, metabolic syndrome occurs when a subject meets three or more of five criteria related to obesity, hypertriglyceridemia, low HDL cholesterol, high blood pressure, and high fasting glucose.

The term “selective modulator of PPARγ” as used herein is defined as any natural or synthetic substance capable of binding to a PPARγ nuclear receptor in such a manner that the substance activates the receptor's ability to cause one or more desired biological effects, without also activating (or with substantially reduced activation of) the receptor's ability to cause one or more undesired biological effects. For example, selective modulators of PPARγ suitable for administration to a diabetic patient in the combinations of the present invention include compounds such as Compound 101 that either naturally (or by design, in the case of compound 101) are capable of interacting with the PPARγ binding pocket in a manner that results in the same or substantially the same insulin sensitizing effects attainable from so-called “full agonists” of PPARγ such as rosiglitazone (Avandia) and pioglitazone (Actos), but without, or with substantial mitigation of, the known harmful side effects associated with such full agonists, including, for example, their tendency to promote weight gain, fluid retention, and bone fracture. The term “selective modulator of PPARγ” should thus be understood to exclude substances such as the full agonists of PPAR that are generally understood by persons of ordinary skill in the art as being able to activate substantially the full spectrum of PPARγ effects, while having little if any ability to differentially activate only the beneficial effects of the receptor and not its harmful effects. A specific example of a class of full PPARγ agonists excluded from the present definition of “selective modulator of PPAR” is the thiazolidinedione (TZD) class of PPARγ full agonists. One of the key benefits of a selective PPARγ modulator is that, unlike a full or non-selective PPARγ agonists, administering increasing dosages of a selective modulator of PPARγ to a patient with diabetes can result in an increase in therapeutic benefits over the selected dose range, with significantly reduced if any concomitant increase in harmful side effects. This separation in the dose response curves for beneficial versus harmful effects allows a broad therapeutic window for administration of a selective modulator of PPARγ to a diabetic patient. Selective modulators of PPARγ (also called SPPARM's) are discussed in “Higgins L S, Montzoros C S, “The development of INT131 as a Selective PPARγ Modulator: Approach to a Safer Insulin Sensitizer,” PPAR Research Volume 2008; Article ID 936906; and Zhang F, Lavan B E, Gregoire F M. “Selective Modulators of PPARγ Activity: Molecular Aspects Related to Obesity and Side-Effects,” PPAR Research Volume 2007 Article ID 32696; and Fujimora T, Kimura C, Oe T, Takata Y, Sakuma H, Aramori, I Mutoh S. “A Selective Peroxisome Proliferator-Activated Receptor γ Modulator with Distinct Fat Cell Regulation Properties,” Journal of Pharmacology and Experimental Therapeutics 2006 Vol 318, No 2 pages 863-871. These publications are incorporated by reference herein in their entirety. To the extent references cited within these publications disclose selective PPARγ modulators, as defined herein, such cited references are further understood to be incorporated by reference herein in their entirety.

### 4.2 Combinations Comprising a PPARγ Modulator and an Incretin

The present invention provides combinations comprising a selective PPARγ modulator and an incretin which are useful in the treatment or prevention of diabetes (including pre-diabetes), obesity, or disorders related to diabetes or obesity. The combinations of these agents can produce a more effective treatment with fewer side effects than treatment with either single agent alone. In particular, the combinations eliminate or significantly reduce the side effects of weight gain, fluid retention and decreased bone density. It is believed that the combination of a selective PPAR gamma modulator and an incretin can provide improved metabolic profile, including glucose metabolism, glucose lowering, and lipid metabolism, greater durability of therapeutic benefit, and reduction in side effects to a greater degree than would be expected or predicted from simply adding the known therapeutic effects of one of these agents to the known effects of the other. It is also believed that the combination of a selective PPAR gamma modulator according to the present invention and an incretin can provide a synergistic reduction in side effects, especially as compared to side effects that may occur in a combination therapy with so-called “full agonists” of PPAR gamma and incretins.

### 4.2.1 Selective PPARγ Modulators

A selective PPARγ modulator suitable for use in the combinations of the present invention is the selective PPARγ modulator (2,4-Dichloro-N-[3,5-dichloro-4-(quinolin-3-yloxy)-phenyl]-benzenesulfonamide benzenesulfonate salt), or compound 101 having the general formula (I), or a pharmaceutically acceptable salt, hydrate or polymorph thereof:

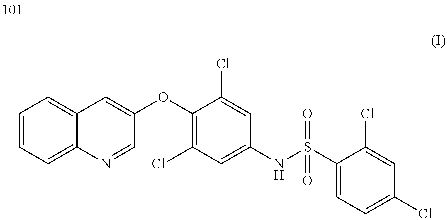

The above selective PPARγ modulator of compound 101 is disclosed, for example, in international patent publication no. WO 01/00579 (corresponding to U.S. Pat. No. 7,041,691), U.S. Pat. Nos. 6,200,995, 6,583,157, 6,653,332, the contents of which are incorporated by reference in their entireties.

An exemplary synthesis of compound 101 is described below. Alternate methods of synthesizing compound 101 will be apparent to those of skill in the art.

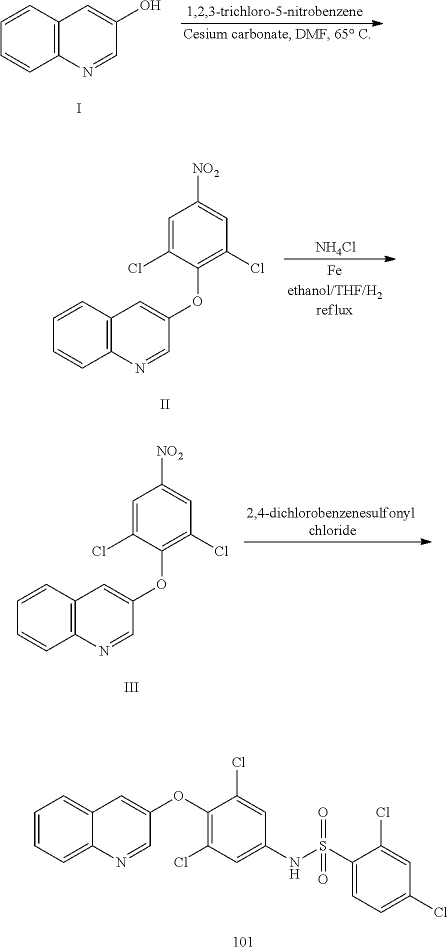

### 3-(2,6-Dichloro-4-nitro-phenoxy)-3,4-dihydro-quinoline (II)

3-Hydroxyquinoline (I) (prepared according to the procedure of Naumann et. al., Synthesis 4:279-281 (1990)) (3 g) and 1,2,3-trichloro-5-nitrobenzene (4.7 g) were dissolved in DMF (80 mL) and heated with cesium carbonate (7.4 g) for 2 h at 60° C. The reaction was poured into ice/water (500 mL). The resulting off-white precipitate was collected by filtration and rinsed with hexane to afford compound II as a solid (6.9 g) suitable for use in the next reaction.

¹H NMR in CDCl₃ δ 8.863 (d, J=2.2 Hz, 1H), 8.360 (s, 2H), 8.106 (d, J=8.6 Hz, 1H), 7.646 (m, 2H), 7.529 (d, J=8.6 Hz, 1H), 7.160 (d, J=2.2 Hz, 1H).

### 3,5-Dichloro-4-(3,4-dihydro-quinolin-3-yloxy)-phenylamine (III)

To a solution of compound II (6.9 g) in ethanol/THF/water (ratio 40:20:10) was added ammonium chloride (3.3 g) and powdered iron (3.4 g). This mixture was heated to reflux for 5 h. The hot mixture was then filtered through Celite and concentrated. The residue was dissolved in ethyl acetate and washed with saturated NaHCO₃ solution followed by water and then brine. The solution was dried over magnesium sulfate and concentrated to afford compound III as an off-white solid (5.6 g).

¹H NMR in (DMSO) δ 8.846 (d. J=2.9 Hz, 1H). 8.010 (m, 1H), 7.915 (m, 1H), 7.645 (m, 1H), 7.560 (m, 1H), 7.401 (d, J=2.9 Hz, 1H), 6.778 (s, 2H), 5.762 (s, 2H).

### 2,4-Dichloro-N-[3,5-dichloro-4-(quinolin-3-yloxy)-phenyl]-benzenesulfonamide (101)

Treatment of the aniline III with 2,4-dichlorobenzenesulfonyl chloride according to conventional methods gave compound 101

¹H NMR (d₆-acetone) δ 9.9 (1H, br s), 8.794 (1H, d, J=2.9 Hz), 8.23 (1H, d, J=8.4 Hz), 8.035 (1H, br d, J=8.4 Hz), 7.793 (1H, d, J=1.5 Hz), 7.78 (1H, m), 7.62-7.70 (2H, m), 7.57 (1H, td, J=6.8, 1.2 Hz), 7.476 (2H, s), 7.364 (1H, d, J=2.6 Hz). MS (M-H) 511.0.

Using methods similar to Lehmann et al., J. Biol. Chem. 270:12953-12956 (1995), compound 101, prepared according to the synthesis method described above, exhibited an IC₅₀ of less than 1 μM in a PPARγ ligand binding assay utilizing [³H]-BRL 49653 as the radioligand.

The pharmaceutically acceptable salts and polymorphs of compound 101 are disclosed in U.S. Pat. Nos. 6,583,157, 6,770,648, 6,653,332, 7,041,691, and 7,223,761, the contents of which are incorporated by reference in their entireties. Each salt of compound 101 can be made from a preparation of compound 101.

The pharmaceutically acceptable salts of compound 101 include but are not limited to benzenesulfonate, hydrochloride salt, or p-toluenesulfonate salt forms of compound 101. Such salt forms are described in detail in U.S. Pat. No. 7,233,761, the contents of which are incorporated by reference in its entirety.

The besylate salt of compound 101 was synthesized from 2,4-dichloro-N-[3,5-dichloro-4-quinolin-3-yloxy)phenyl]-benzenesulfonamide HCl prepared as described in detail in U.S. Pat. No. 7,223,671, the contents of which is incorporated by reference in its entirety. The hydrochloride salt 2,4-dichloro-N-[3,5-dichloro-4-quinolin-3-yloxy)phenyl]-benzenesulfonamide HCl was converted to the besylate salt, via the free base, using a sodium bicarbonate/ethyl acetate biphasic reaction solution. Separation of the organic layer followed by solvent exchange with ethanol precipitated the besylate salt (6) of compound 101 in 84% yield. Starting from 4-aminoquinoline (2), the overall yield of the besylate salt (6) of compound 101 was 73%.

The benzenesulfonate of such salts of formula (I) is according to formula (II):

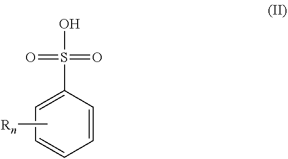

In formula (II), the phenyl ring is optionally substituted with R as described above, and n is any integer from 1 to 5. In certain embodiments, R is heteroalkyl, alkyl or hydrogen, and n is any integer from 1 to 5. In further embodiments, R can be alkyl or hydrogen, and n is any integer from Ito 5. In some embodiments, R is lower alkyl or hydrogen, and n is any integer from 1 to 5. In some embodiments, each R is hydrogen. The besylate salt of compound 101 is provided by formula (III):

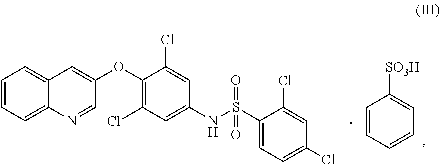

Compound 101 can be synthesized or obtained according to any method apparent to those of skill in the art. In some embodiments, compound 101 is prepared according to the methods described in detail in the examples below and in U.S. Pat. Nos. 6,583,157 and 7,041,691, the contents of which are hereby incorporated by reference in their entireties.

Alternatively, compound 101 can be prepared by isolating a salt of compound 101 as described below and converting such a salt of compound 101 to the neutral form by treatment with an appropriate base. For example, compound 101 can be prepared by isolating the hydrochloride salt of compound 101 by filtration, then converting it to the neutral form by treatment with monobasic sodium carbonate in ethyl acetate, or other suitable base. In such embodiments, the hydrochloride salt of compound 101 can be prepared by any method known to one of skill in the art. For example, the hydrochloride salt of compound 101 can be prepared by reacting 3,5-dichloro-4-(quinolin-3-yloxy)-phenylamine with 2,4-dichlorobenzenesulfonylchloride and hydrochloric acid to yield 2,4-dichloro-N-[3,5-dichloro-4-quinolin-3-yloxy)phenyl]-benzenesulfonamide HCl.

The polymorphs of compound 101 are described in detail in U.S. Pat. No. 7,233,761, the contents of which is incorporated by reference in its entirety.

Each polymorph of the invention can be made from a preparation of compound 101. Solid compound 101 can be dissolved and then crystallized from the solvent mixtures described below to yield the polymorphic forms of the invention. In particular embodiments of the invention, a besylate salt of compound 101 can be dissolved and then crystallized from the solvent mixtures described below to yield the polymorphic forms of compound 101.

In some embodiments, Form I of a besylate salt of compound 101 (2,4-Dichloro-N-[3,5-dichloro-4-(quinolin-3-yloxy)-phenyl]-benzenesulfonamide benzenesulfonate salt) is used. In such embodiments, the Form I polymorph of the besylate salt of compound 101 may have a melting point of about 180° C. or greater. In a particular embodiment, the Form I polymorph may have a melting point between about 180 and 200° C. When an exemplary Form I polymorph was examined by differential scanning calorimetry according to the methods described in the examples below, it had an endotherm at between about 186.3° C. and about 189.5° C. and an enthalpy of fusion of between about 81.5 J/g and about 89.9 J/g. For example, particular Form I polymorphs of the invention have major X-ray powder diffraction pattern peaks at 7.0, 19.5, 22.0, 24.0, 24.5 and 28° 2θ using Cu Kα radiation. In certain embodiments, the Form I polymorph can have major X-ray powder diffraction pattern peaks at one, two, three, four, five or six of the X-ray powder diffraction pattern peaks at 7.0, 19.5, 22.0, 24.0, 24.5 and 28° 2θ using Cu Kα radiation. In further embodiments, the Form I polymorph can have both a melting point between about 186 and 200° C. and major X-ray powder diffraction pattern peaks at one, two, three, four, five or six of the X-ray powder diffraction pattern peaks at 7.0, 19.5, 22.0, 24.0, 24.5 and 28° 20 using Cu Kα radiation. In still further embodiments, the Form I polymorph can have major infrared absorbance peaks at one, two, three, four, or five of the infrared absorbance peaks at 1567, 1461, 913, 895, and 881 cm⁻¹.

Form I of the besylate salt of compound 101 can be made by any method of making Form I apparent to those of skill in the art. For example, Form I can be crystallized from ethanol solutions of compound 101 and a hydrate of benzenesulfonic acid. Preferably, an ethanol solution of benzenesulfonic acid hydrate (Aldrich) can be added to solid compound 101 under heat to complete solution; cooling the solution yields Form I. Form I can also be crystallized from solutions of ethyl acetate and ethanol as described in the U.S. Pat. No. 7,233,761, the contents of which is incorporated by reference in its entirety.

In some embodiments, Form II of the besylate salt of compound 101 (2,4-Dichloro-N-[3,5-dichloro-4-(quinolin-3-yloxy)-phenyl]-benzenesulfonamide benzenesulfonate salt) is used. In some embodiments, the Form II polymorph of the besylate salt of compound 101 can have a melting point of about 230° C. or greater. In some embodiments, the Form II polymorph can have a melting point between about 230 and 240° C. An exemplary Form II of the besylate salt of compound 101 displayed surprising stability and had a melting temperature of about 233° C. When an exemplary Form II polymorph was examined by differential scanning calorimetry according to the methods in the examples below, it had an endotherm at about 233.7° C. and an enthalpy of fusion of about 98.9 J/g. For example, particular Form II polymorphs can have major X-ray powder diffraction pattern peaks at 15, 19, 20.5, 23.5, 24.5, 25, 26.5, 29.5 and 30.5° 2θ using Cu Kα radiation. In certain embodiments, the Form II polymorph can have major X-ray powder diffraction pattern peaks at one, two, three, four, five, six, seven or eight of the X-ray powder diffraction pattern peaks at 15, 19, 20.5, 23.5, 24.5, 25, 26.5, 29.5 and 30.5° 2θ using Cu Kα radiation. In certain embodiments, the Form II polymorph of the invention can have both a melting point between about 230 and 240° C. and major X-ray powder diffraction pattern peaks at one, two, three, four, five, six, seven or eight of the X-ray powder diffraction pattern peaks at 15, 19, 20.5, 23.5, 24.5, 25, 26.5, 29.5 and 30.5° 2θ using Cu Kα radiation. In further embodiments, the Form II polymorph can have major infrared absorbance peaks at one, two, three, four, or five of the infrared absorbance peaks at 1573, 1469, 1459, 912, and 859 cm⁻¹.

Form II of the besylate salt of compound 101 can be made by any method apparent to those of skill in the art to make Form II based upon the teachings herein. For example, Form II can be crystallized from solutions of ethyl acetate and ethanol as described in detail in U.S. Pat. No. 7,233,761, the contents of which is incorporated by reference in its entirety. Preferably, Form II of the besylate salt of compound 101 can be prepared by adding an ethanol solution of benzenesulfonic acid to solid compound 101 under heat. The reaction suspension can be stirred under heat, then cooled under further stirring, which yields Form II of the besylate salt of compound 101.

Other selective PPARγ modulators that can be used in the combination of the invention are known in the literature. Illustrative examples of selective modulators of PPARγ are GW0072 (Glaxo Welcome); halofenate and its enantiomer metaglidasen (Metabolex); PA-082 (Roche); angiotensin receptor blockers (ARBs) including telmisartan, losartan, eprosartan, valsartan, and candesartan; and YM440. The foregoing, and other examples of selective PPARγ modulators, as defined herein, are described and referenced in Zhang et al., and Higgins et al., cited above.

Incretins

The incretin can be any compound that is a gastrointestinal hormone, or its mimetic, that causes an increase in the amount of insulin released from the beta cells of the islets of Langerhans in response to food intake. Examples of incretins are described, for example, in Ding X, Saxena N K, Lin S, Gupta N A, Anania F A. “Exendin-4, a glucagon-like protein-1 (GLP-1) receptor agonist, reverses hepatic steatosis in ob/ob mice”, Hepatology. 2006; 43(1):173-81; Tushuizen M E, Bunck M C, Pouwels P J, van Waesberghe J H, Diamant M, Heine R J, “Incretin mimetics as a novel therapeutic option for hepatic steatosis”, Liver Int. 2006; 26(8):1015-7. Fowler M J. “Diabetes Treatment, Part 3: Insulin and Incretins. Clinical Diabetes 2008; 26(1):35-39; Drucker D J. “Enhancing incretin action for the treatment of type 2 diabetes.” Diabetes Care 2003; 26:2929-2940; Turton M D, O'Shea D, Gunn I, Beak S A, Edwards C M, Meeran K, Choi S J, Taylor G M, Heath M M, Lambert P D, Wilding J P, Smith D M, Ghatei M A, Herbert J, Bloom S R “A role for glucagon-like peptide-1 in the central regulation of feeding.” Nature 1996; 379:69-72; Nauck M A, Bailer B, Meier J J. “Gastric inhibitory polypeptide and glucagon-like peptide-1 in the pathogenesis of type 2 diabetes.” Diabetes 2004 53 (Suppl. 3):S190-196, the contents of which are hereby incorporated by reference in their entirety.

In one embodiment, the incretin is selected from the group consisting of a glucagone-like peptide-1 (GLP-1) receptor agonist and glucose-dependent insulinotropic peptide (GIP) receptor agonist. In certain embodiments, the incretin is selected from the group consisting of exenatide, a long-acting-release (LAR) variant of exenatide, liraglutide, taspoglutide, CJC-1131, LY307161 SR, and AVE0010/ZP10.

In certain embodiments, the incretin is selected from the group consisting of glucagone-like peptide-1 (GLP-1) receptor agonist and glucose-dependent insulinotropic peptide (GIP) receptor agonist.

In some embodiments, the incretin is exenatide (Byetta) or Byetta LAR. Exenatide is described, for example, in U.S. Pat. Nos. 5,424,286; 6,902,744; 7,297,761, and others, the contents of each of which is herein incorporated by reference in its entirety.

In one embodiment, the incretin is liraglutide (also called NN-2211 and [Arg34, Lys26]-(N-epsilon-(gamma-Glu(N-alpha-hexadecanoyl))-GLP-1 (7-37)), includes the sequence HAEGTFTSDVSSYLEGQAAKEFIAWKVRGRG and is available from Novo Nordisk (Denmark) or Scios (Fremont, Calif. USA). See, e.g., Elbrond et al., 2002, Diabetes Care. August; 25(8):1398404; Agerso et al., 2002, Diabetologia. February; 45(2):195-202). In another embodiment, the incretin is taspoglutide (CAS Registry No. 275371-94-3). See, for example, U.S. Pat. No. 7,368,427.

In another embodiment, the incretin is CJC-1131, which is a GLP-1 analogue that consists of a DPP-IV-resistant form of GLP-1 joined to a reactive chemical linker group that allows GLP-1 to form a covalent and irreversible bond with serum albumin following subcutaneous (SC) injection. See, Kim et al., 2003, Diabetes 52:751-759. CJC-1131 is available from ConjuChem (Montreal, Quebec, Canada).

In another embodiment, the incretin is LY307161 SR, which is sustained release formulation of a GLP-1 analog suitable for once daily administration. See, J. Pharm Sci. 2005 December; 94(12):2749-63. Doyle B L, Polio M J, Pekar A H, Roy M L, Thomas B A, Brader M L. Curr Med. Chem. 2003 November; 10(22):2471-83; and Holz G G, Chepumy, O G. Glucagon-like Peptide-1 Synthetic Analogs: New Therapeutic Agents for use in the Treatment of Diabetes mellitus. The foregoing references are incorporated by reference in their entirety.

In another embodiment, the incretin is AVE0010/ZP10, which is a modified exendin-4, having six additional lysine residues, available from Aventis/Zealand Pharma. See Preclinical pharmacology of the new GLP-1 receptor agonist AVE0010. Werner U. Ann Endocrinol (Paris), 2008 April; 69(2):164-5, Epub 2008 Apr. 16; Glucagon-like Peptide 1 Receptor Agonist ZP10A Increases Insulin mRNA Expression and Prevents Diabetic Progression in db/db mice, Thorkildsen C, Neve S, Larsen B D, Meier E, Petersen J S. J Pharmacol Exp Ther. 2003 November; 307(2):490-6. Epub 2003 Sep. 15. The foregoing references are incorporated by reference in their entirety.

### 4.2.2 Pharmaceutical Compositions

The combinations of the present invention may be in the form of a pharmaceutical composition that further comprises a pharmaceutically acceptable diluent, excipient or carrier.

The pharmaceutical compositions may conveniently be presented in unit dosage form and may be prepared by any of the methods well known in the art of pharmacy. In general, the pharmaceutical compositions are prepared by uniformly and intimately bringing the combinations into association with a liquid carrier or a finely divided solid carrier or both, and then, if necessary, shaping the product into the desired formulation. In the pharmaceutical composition, each of the active ingredient is included in an amount sufficient to produce the desired effect upon the process, condition or disease to be modulated, prevented, or treated.

The pharmaceutical compositions containing the combinations may be in a form suitable for oral use, for example, as tablets, troches, lozenges, aqueous or oily suspensions, dispersible powders or granules, emulsions, hard or soft capsules, or syrups, solutions, or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the combinations in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452, and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Formulations and dosages of the invention may be provided in the form of formulations suitable for parenteral (including intravenous, intramuscular and subcutaneous) administration. Also described herein are formulations and dosages useful in alternative delivery routes, including oral, nasal, buccal, sublingual and pulmonary.

Compounds useful in the invention can be provided as parenteral compositions for injection or infusion. Generally, they can, for example, be suspended in an inert oil, suitably a vegetable oil such as sesame, peanut, olive oil, or other acceptable carrier. Preferably, they are suspended in an aqueous carrier, for example, in an isotonic buffer solution at a pH of about 3.0 to about 7.0, more specifically from about 4.0 to 6.0, and preferably from about 4.0 to about 5.0. These compositions may be sterilized by conventional sterilization techniques, or may be sterile filtered. The compositions may contain pharmaceutically acceptable auxiliary substances as required to approximate physiological conditions, such as pH buffering agents. Useful buffers include for example, sodium acetate/acetic acid buffers. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions.

Oily suspensions may be formulated by suspending the combinations in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the combinations in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example, liquid paraffin, or mixtures of these. Suitable emulsifying agents may be naturally-occurring gums, for example, gum acacia or gum tragacanth; naturally-occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The pharmaceutical compositions may also be administered in the form of suppositories suitable for rectal administration of the drug. These compositions can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

For topical use, creams, ointments, jellies, solutions or suspensions, etc., containing the salts or polymorphs of the present invention may be employed. As used herein, topical application is also meant to include the use of mouth washes and gargles.

The pharmaceutical compositions may further comprise other therapeutically active compounds known to one skilled in the art to be useful in the treatment or prevention of the above mentioned pathological conditions.

### 4.3 Methods of Treatment Using a Selective PPARγ Modulator and an Incretin

The present invention provides the use of a selective PPARγ modulator or a pharmaceutically acceptable salt, hydrate, or polymorph thereof, and an incretin as a combination therapy for the treatment of various disorders such as diabetes, obesity, disorders related to diabetes or obesity.

Thus, the present invention provides methods of treating diabetes, obesity or disorders related to diabetes or obesity by administering to a subject in need thereof, a therapeutically effective amount of a selective PPARγ modulator, e.g., a compound of formula (I):

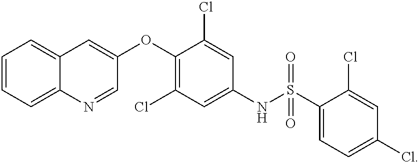

or a pharmaceutically acceptable salt, hydrate or polymorph thereof; and a therapeutically effective amount of an incretin.

The subject can be an animal such as, for example, a mammal, including, but not limited to, a primate (e.g., a human), a cow, a sheep, a goat, a horse, a dog, a cat, a rabbit, a rat, a mouse and the like. In certain embodiments, the subject is human.

The combinations of the present invention are useful for the treatment of diabetes. The diabetes may be due to any cause, whether genetic or environmental.

Diabetes treatable with the compositions of the present invention include T2DM and pre-diabetes. T2DM, or insulin-independent diabetes (i.e., non-insulin-dependent diabetes mellitus), often occurs in the face of normal, or even elevated levels of insulin and appears to be the result of the inability of tissues to respond appropriately to insulin. The development of T2DM is related to obesity; most Type 2 diabetics are also obese. The combinations of the present invention are useful for treating both T2DM, as well as for preventing the onset or progression of T2DM in individuals diagnosed with T2DM or pre-diabetes. The combinations of the present invention are also useful for treating and/or preventing gestational diabetes mellitus. The combinations of the present invention are also useful for treating and/or preventing progression of early stage Type 1 diabetes.

In some embodiments, diabetes can be characterized by a fasting plasma glucose level of greater than or equal to 126 mg/dl. In some embodiments, a diabetic subject can have a fasting plasma glucose level of greater than or equal to 126 mg/dl. In some embodiments, prediabetes can be characterized by an impaired fasting plasma glucose (FPG) level of greater than or equal to 110 mg/dl and less than 126 mg/dl; or impaired glucose tolerance; or insulin resistance. In some embodiments, a prediabetic subject can be a subject with impaired fasting glucose (a fasting plasma glucose (FPG) level of greater than or equal to 110 mg/dl and less than 126 mg/dl); or impaired glucose tolerance (a 2 hour plasma glucose level of >140 mg/dl and <200 mg/dl); or insulin resistance, resulting in an increased risk of developing diabetes.

The combinations of the present invention are useful for the treatment of obesity. The term “obesity” as used herein is a condition in which there is an excess of body fat. In some embodiments, obesity can be defined based on the Body Mass Index (BMI), which is calculated as body weight per height in meters squared (kg/m²). In some embodiments, obesity can refer to a condition whereby an otherwise healthy subject has a Body Mass Index (BMI) greater than or equal to 30 kg/m², or a condition whereby a subject with at least one co-morbidity has a BMI greater than or equal to 27 kg/m². In some embodiments, an obese subject can be an otherwise healthy subject with a Body Mass Index (BMI) greater than or equal to 30 kg/m² or a subject with at least one co-morbidity with a BMI greater than or equal to 27 kg/m².

The combinations of the present invention are useful for the treatment of disorders related to diabetes or obesity. In certain embodiments, the disorders related to diabetes or obesity is selected from the group consisting of hyperglycemia, prediabetes, impaired glucose tolerance, impaired fasting glucose, dyslipidemia, hyperlipidemia, hypertriglyceridemia, hypercholesterolemia, low HDL levels, high LDL levels, atherosclerosis, hypertension, sleep apnea, polycystic ovarian syndrome, nonalcoholic steatohepatitis and metabolic syndrome.

The diabetes-related disorders herein can be any disorders associated with, caused by, or result from diabetes. Examples of diabetes-related disorders include but are not limited to hyperglycemia, impaired glucose tolerance, insulin resistance, obesity, lipid disorders, dyslipidemia, hyperlipidemia, hypertriglyceridemia, hypercholesterolemia, low HDL levels, high LDL levels, atherosclerosis and its sequelae, vascular restenosis, irritable bowel syndrome, inflammatory bowel disease, including Crohn's disease and ulcerative colitis, other inflammatory conditions, pancreatitis, abdominal obesity, neurodegenerative disease, retinopathy, neoplastic conditions, adipose cell tumors, adipose cell carcinomas, such as liposarcoma, prostate cancer and other cancers, including gastric, breast, bladder and colon cancers, angiogenesis, Alzheimer's disease, psoriasis, high blood pressure, Metabolic Syndrome, ovarian hyperandrogenism (polycystic ovary syndrome), nonalcoholic steatohepatitis and other disorders where insulin resistance is a component, such as sleep apnea. The combinations of the present invention are particularly useful for the treatment of hyperglycemia, impaired glucose tolerance, obesity, dyslipidemia, hyperlipidemia, hypertriglyceridemia, hypercholesterolemia, low HDL levels, atherosclerosis, and metabolic syndrome.

The combinations of the present invention are useful for the treatment of obesity-related disorders. The obesity-related disorders herein can be any disorders associated with, caused by, or result from obesity. Examples of obesity-related disorders include but are not limited to obesity, diabetes, overeating, binge eating, and bulimia, hypertension, elevated plasma insulin concentrations and insulin resistance, dyslipidemia, hyperlipidemia, endometrial, breast, prostate, kidney and colon cancer, osteoarthritis, obstructive sleep apnea, gallstones, heart disease, abnormal heart rhythms and arrythmias, myocardial infarction, congestive heart failure, coronary heart disease, sudden death, stroke, polycystic ovary disease, craniopharyngioma, Prader-Willi Syndrome. Frohlich's syndrome, GH-deficient subjects, normal variant short stature, Turne's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, lower back pain, gallbladder disease, hyperuricemia, gout, and kidney cancer, and increased anesthetic risk. The combinations of the present invention are also useful to treat Alzheimer's disease.

The combinations of the present invention are also useful for the treatment or prevention of metabolic syndrome. “Metabolic syndrome” can be defined as described in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following disorders: abdominal obesity, hypertriglyceridemia, low HDL cholesterol, high blood pressure, and high fasting plasma glucose. The criteria for these are defined in ATP-III. Treatment of metabolic syndrome refers to the administration of the combinations of the present invention to a subject with metabolic syndrome or a subject that has developed two of the disorders that define metabolic syndrome, but has not yet developed three or more of the disorders that define metabolic syndrome.

### 4.3.1 Routes of Administration and Dosage

The particular selective PPARγ modulator and the incretin can be administered in a single pharmaceutical dosage formulation which contains the particular selective PPARγ modulator and the incretin, or each agent be administered in its own separate pharmaceutical dosage formulation. Where separate dosage formulations are used, the individual active ingredient can be administered at essentially the same time, i.e., concurrently, or at separately staggered times, i.e. sequentially prior to or subsequent to the administration of the other active ingredient. The instant methods are therefore to be understood to include all such regimes of simultaneous or non-simultaneous treatment.

Administration in these various ways is suitable for the present compositions as long as the beneficial pharmaceutical effect of the combination of the particular selective PPARγ modulator and the incretin is realized by the subject at substantially the same time. Such beneficial effect is preferably achieved when the target blood level concentrations of each active ingredient are maintained at substantially the same time. It is preferred that the combination of the particular selective PPARγ modulator and the incretin be co-administered concurrently on a once-a-day dosing schedule; however, varying dosing schedules, such as twice or more times per day, is also encompassed herein. A single oral dosage formulation comprised of both active ingredient in the combination is preferred. A single dosage formulation will provide convenience for the patient, which is an important consideration especially for patients with diabetes, metabolic syndrome, or obese patients who may be in need of multiple medications. However, not all incretins may be administered orally at the present time, and therefore, the invention encompasses methods of administration where a PPARγ modulator and an incretin are administered through different routes.

Thus, in one embodiment, a selective PPARγ modulator may be administered orally, while an incretin may be administered by an injection or through other routes of administration.

Pharmaceutical compositions of the particular selective PPARγ modulator and incretin, either individually or in combination, may be prepared by methods well known in the art as described above, e.g., by means of conventional mixing, dissolving, granulation, dragee-making, levitating, emulsifying, encapsulating, entrapping, lyophilizing processes or spray drying.

The dosage of the particular selective PPARγ modulator and the incretin can be determined in accordance with the judgment of one of skill in the art. In the case where a single composition is employed, a suitable dosage range can be, e.g. from about 0.001 mg/kg to about 100 mg/kg of each compound in the composition per day, preferably from about 0.01 mg to about 2000 mg per day. For oral administration, the compositions can be provided in the form of tablets containing from 0.01 mg to 2,000 mg, e.g. 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850, 1,000 and 2,000 milligrams of each compound for the symptomatic adjustment of the dosage to the subject to be treated. This dosage regimen may be adjusted to provide the optimal therapeutic response. For injections, the compositions can be provided in the form of aqueous formulations.

The particular selective PPARγ modulator in the combinations of the present invention can be administered at a daily dosage of from about 0.001 mg to about 1 mg per kilogram of animal body weight, preferably given as a single daily dose or in divided doses two to six times a day, or in sustained release form. For humans, the total daily dosage can be from about 0.01 mg to about 100 mg, preferably from about 0.1 mg to about 20 mg. This dosage regimen may be adjusted to provide the optimal therapeutic response.

The invention also includes preferred dosages for incretins when given by injection, and when given by other routes. Thus, incretin formulations may be prepared for the administration by injection and may include from about 0.1 to about 5,000 μg of incretin per kilogram, given one to three times per day. The dosage can be varied substantially based on the particular incretin selected, and depending upon whether the dosages are intended for daily administration or for administration spaced over longer intervals (such as once weekly). Typically, for the patient with diabetes who weighs in the range from about 70 kilograms (average for the type 1 diabetic) to about 90 kilograms (average for the type 2 diabetic), for example, this will result in the total administration of about 2 to about 60,000 μg of incretin per day in single or divided doses. If administered in divided doses, the doses are preferably administered two or three times per day, and more preferably, two times per day. This dosage regimen may be adjusted to provide the optimal therapeutic response.

The weight ratio of the particular selective PPARγ modulator and the incretin may be varied and will depend upon the effective dose of each ingredient. Generally, an effective dose of each will be used.

Incretins may be administered parenterally, more preferably by injection, for example, by peripheral or subcutaneous injection. Preferably, about 2-100,000 μg of the incretin is administered per day. More preferably, about 1-30 μg to about 500 μg, or about 1-30 μg to about 50 μg of the incretin is administered per day. Most preferably, depending upon the weight of the subject and the potency of the compound administered, about 3 μg to about 50 μg of the incretin is administered per day. Preferred doses based upon patient weight for compounds having approximately the potency of incretin range from about 0.005 μg/kg per dose to about 0.2 μg/kg per dose. More preferably, doses based upon patient weight for compounds having approximately the potency of incretin range from about 0.02 μg/kg per dose to about 0.1 μg/kg per dose. Most preferably, doses based upon patient weight for compounds having approximately the potency of incretin range from about 0.05 μg/kg per dose to about 0.1 μg/kg per dose. These doses are administered from 1 to 4 times per day, preferably from 1 to 2 times per day. Doses of incretins will normally be lower if given by continuous infusion. Doses of incretins will normally be higher if given by non-injection methods, such as oral, buccal, sublingual, nasal, pulmonary or skin patch delivery.

Depending on the disease to be treated and the subject's condition, the particular selective PPARγ modulator and the incretin may be administered by oral, parenteral (e.g., intramuscular, intraperitoneal, intravenous, ICV, intracisternal injection or infusion, subcutaneous injection, or implant), inhalation spray, nasal, vaginal, rectal, sublingual, or topical routes of administration and may be formulated, alone or together, in suitable dosage unit formulations containing conventional non-toxic pharmaceutically acceptable diluents, excipients or carriers appropriate for each route of administration. When the particular selective PPARγ modulator and the incretin are administered separately, they may be administered by different routes.

It will be understood, however, that the specific dose level and frequency of dosage for any particular patient may be varied and will depend upon a variety of factors including the activity of the specific polymorph employed, the metabolic stability and length of action of that polymorph, the age, body weight, general health, sex, diet, mode and time of administration, rate of excretion, drug combination, the severity of the particular condition, and the host undergoing therapy.

### 4.3.2 Combination Therapy

The combinations of the present invention can be further combined with other compounds having related utilities to treat diabetes, obesity or disorders related to diabetes or obesity. In many instances, administration of the subject compounds or compositions in conjunction with these alternative agents enhances the efficacy of such agents. Accordingly, in some instances, the present combinations, when combined or administered in combination with, e.g., additional anti-diabetic agents, can be used in dosages which are less than the expected amounts when used alone, or less than the calculated amounts for combination therapy.

For example, suitable agents for combination therapy include those that are currently commercially available and those that are in development or will be developed. Exemplary agents useful in the treatment of diabetes, obesity or disorders related to diabetes or obesity include, but are not limited to: (a) anti-diabetic agents such as insulin, sulfonylureas (e.g., meglinatide, tolbutamide, chlorpropamide, acetohexamide, tolazamide, glyburide, glipizide and glimepiride), biguanides, e.g., metformin (Glucophage®), α-glucosidase inhibitors (acarbose) (b) β₃ adrenergic receptor agonists, leptin or derivatives thereof and neuropeptide Y antagonists; (c) bile acid sequestrants (e.g., cholestyramine and colestipol), HMG-CoA reductase inhibitors, e.g., statins (e.g., lovastatin, atorvastatin, fluvastatin, pravastatin and simvastatin), nicotinic acid (niacin), fibric acid derivatives (e.g., gemfibrozil and clofibrate) and nitroglycerin.

### 4.4 Kits

The present invention further provides kits comprising a particular selective PPARγ modulator and an incretin. The particular selective PPARγ modulator and the incretin are described in detail above.

In certain embodiments, the kits comprise a therapeutically effective amount of a compound of formula (I):

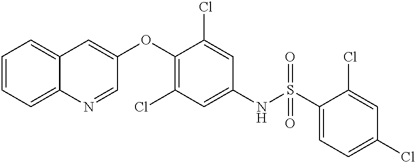

or a pharmaceutically acceptable salt, hydrate or polymorph thereof; and a therapeutically effective amount of an incretin.

In certain embodiments, the kits comprise two separate pharmaceutical compositions: a first unit dosage form comprising a therapeutically effective amount of a particular selective PPARγ modulator, i.e. compound 101 or a pharmaceutically acceptable salt, hydrate, or polymorph thereof, and a pharmaceutically acceptable carrier or diluent in a first unit dosage form, and a second unit dosage form comprising a therapeutically effective amount of an incretin, and a pharmaceutically acceptable carrier or diluent in a second unit dosage form.

In some embodiments, the kits further comprises a container. Such kits are especially suited for the delivery of solid oral forms such as tablets or capsules. Such kits may include a number of unit dosages. Such kits can include a card having the dosages oriented in the order of their intended use. An example of such a kit is a “blister pack.” Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered.

In some embodiments, the kits further comprise a label or labeling with instruction for using the kits. For example, the label of labeling can provide dosage information and specific methods of administration for the particular selective PPARγ modulator and the incretin.

### 5. EXAMPLES

### 5.1 Example 1

### Combination Therapy Using Compound 101 and Exenatide

This example illustrates combination therapy of compound 101 and exenatide, wherein compound 101 is administered by oral administration and exenatide is administered by injection.

Patients having NIDDM (T2DM) are selected for therapy.

Compound 101 is orally administered in a dosage of 0.10 to 10 milligrams once or twice daily, more typically 1 mg daily. Exenatide is injected in a dosage of 5 μg to 50 μg twice per day. For infants or children the doses suggested are lowered in a linear fashion based on body weight or surface area.

One third of the patients are administered exenatide by injection twice per day. One third of the patients are orally administered compound 101 daily. The remaining third of the patient population is orally administered compound 101 daily and is administered exenatide by injection twice per day.

The patients are monitored for improvement in the manifestations of the disease and for side effects.

### 5.2 Example 2

### Combination Therapy Using Compound 101 and Liraglutide

This example illustrates combination therapy of compound 101 and liraglutide, wherein compound 101 is administered by oral administration and liraglutide is administered by injection.

Patients having NIDDM (T2DM) are selected for therapy. The patients weigh between 70-100 kilograms.

Compound 101 is orally administered in a dosage of 0.10 to 10 milligrams once daily, more typically 1 mg once daily. Liraglutide is injected subcutaneously in a dosage of 0.1 to 3 mg once per day. For infants or children the doses suggested are lowered in a linear fashion based on body weight or surface area.

One third of the patients are administered liraglutide by injection daily. One third of the patients are orally administered compound 101 daily. The remaining third of the patient population is orally administered compound 101 daily and is administered liraglutide by injection once daily.

The patients are monitored for improvement in the manifestations of the disease and for side effects.

### 5.1 Example 3

### Combination Therapy Using Compound 101 and Exenatide LAR (Long-Acting-Release)

This example illustrates combination therapy of compound 101 and exenatide LAR, wherein compound 101 is administered by oral administration and exenatide LAR is administered by injection.

Patients having NIDDM (T2DM) are selected for therapy.

Compound 101 is orally administered in a dosage of 0.10 to 10 milligrams once daily, more typically 1 mg once daily. Exenatide LAR is injected subcutaneously in a dosage of 0.1 to 5 mg once per week. For infants or children the doses suggested are lowered in a linear fashion based on body weight or surface area.

One third of the patients are administered exenatide LAR by injection once per week. One third of the patients are orally administered compound 101 daily. The remaining third of the patient population is orally administered compound 101 daily and is administered exenatide LAR by injection once per week.

The patients are monitored for improvement in the manifestations of the disease and for side effects.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A pharmaceutical composition comprising a compound of formula (I): 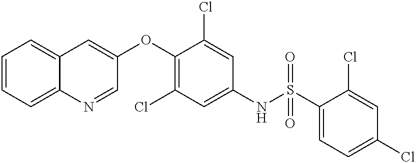
or a pharmaceutically acceptable salt, hydrate or polymorph thereof; and an incretin.

2. The pharmaceutical composition of claim 1, wherein the pharmaceutically acceptable salt of the compound is a benzenesulfonate salt, a hydrochloride salt or a p-toluenesulfonate salt of the compound.

3. The pharmaceutical composition of claim 1, wherein the incretin is a glucagone-like peptide 1 (GLP-1) receptor agonist.

4. The pharmaceutical composition of claim 3, wherein said glucagone-like peptide 1 (GLP-1) receptor agonist is selected from the group consisting of exenatide, a long-acting-release (LAR) variant of exenatide, liraglutide, taspoglutide, CJC-1131, LY307161 SR, and AVE0010/ZP10.

5. The pharmaceutical composition of claim 1, wherein the pharmaceutical composition further comprises a pharmaceutically acceptable diluent, excipient or carrier.

6. A method of treating a condition selected from the group consisting of diabetes, obesity, or a disorder related to diabetes or obesity, comprising administering to a subject in need thereof a therapeutically effective amount of a compound of formula (I): 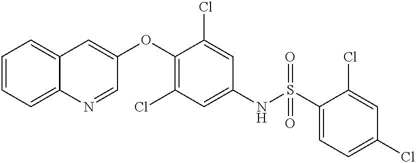
or a pharmaceutically acceptable salt, hydrate or polymorph thereof; and a therapeutically effective amount of an incretin.

7. The method of claim 6 wherein said condition is selected from the group consisting of diabetes and obesity.

8. The method of claim 6 wherein said disorder related to diabetes or obesity is selected from the group consisting of hyperglycemia, prediabetes, impaired glucose tolerance, impaired fasting glucose, dyslipidemia, hyperlipidemia, hypertriglyceridemia, hypercholesterolemia, low HDL levels, high LDL levels, atherosclerosis, hypertension, sleep apnea, polycystic ovarian syndrome, and Metabolic Syndrome.

9. The method of claim 6, wherein said compound and said incretin are administered concurrently.

10. The method of claim 6, wherein said compound and said incretin are administered sequentially.

11. A pharmaceutical composition comprising a selective modulator of PPARγ and an incretin.

12. A method of treating a condition selected from the group consisting of diabetes, obesity, or a disorder related to diabetes or obesity, comprising administering to a subject in need thereof a therapeutically effective amount of a selective PPARγ modulator and an incretin.
